(19) United States
(12) Patent Application Publication
(10) Pub. No.: US 2009/0076446 A1
(43) Pub. Date: Mar. 19, 2009

(54) ADJUSTABLE CATHETER FOR DILATION IN THE EAR, NOSE OR THROAT

(75) Inventors: Victor A. Dubuclet, IV, Dallas, TX (US); Michael L. Dollar, Garland, TX (US)

Correspondence Address: CARSTENS & CAHOON, LLP, P O BOX 802334, DALLAS, TX, 75380, US

(73) Assignee: QUEST MEDICAL, INC., Allen, TX (US)

(21) Appl. No.: 11/855,976

(22) Filed: Sep. 14, 2007

(51) Int. Cl. A61M 25/10

(52) U.S. Cl. 604/96.01

(57) ABSTRACT

The improved balloon catheter includes a first tubular segment, which has multiple zones of differing malleability along its length. The catheter further includes a coaxially aligned hypotube formed of a malleable material, which is positioned within the lumen of the first tubular segment and extends from the distal end of the first tubular segment. In one embodiment the hypotube runs the length of the first tubular segment; while in another embodiment, the hypotube runs along only a portion of the length of the first tubular segment. A portion of the outer circumferential surface of the hypotube is permanently affixed and sealed to an inner circumferential surface of the first tubular segment in the vicinity of the distal end of the first tubular segment. The outer circumference of the distal end of the first tubular segment is gently tapered along its length so as to smoothly transition to the circumference of the hypotube. The hypotube terminates with an atraumatic tip and includes a balloon dilator affixed near the tip. An aperture near the tip of the hypotube fluidly connects the interior of the balloon with the lumen of the hypotube enabling the balloon to selectively expand and contract.

### BACKGROUND OF THE INVENTION

1. Technical Field of the Invention

The present invention relates to surgical balloon catheters and methods for using such catheters for treating paranasal sinus airways and methods for using such catheters for treating paranasal sinuses.

2. Description of the Related Art

In order to fully understand this invention, it is important to consider the anatomy of the sinus system. The sinus system consists of many different pathways, called ducts or ostia, which allow mucus, air and other substances to drain and flow through the system. Inflammation can occur in the tissues that make up the ducts and ostia, causing them to swell and block the normal flow. Inflammation may be caused by allergies, noxious agents, nasal polyps, and other factors. Over time there can be a pathologic increase in inflamed tissue causing permanent disruption in the flow through the sinus system. Obstruction of the narrow ducts and ostia between the paranasal sinuses and nasal cavity develops, resulting in a vicious cycle of increased secretions, edema and ultimately complete blockage of the sinus pathways. The state of chronic sinus inflammation is called sinusitis. Sinusitis can both be caused by and can cause a narrowing of the sinus ostia.

Treatment with antibiotics, corticosteroids in nasal sprays or systematically may result in effective resolution of sinusitis. However, some patients become resistant to oral medical treatment and surgical intervention becomes necessary.

Modern sinus surgery is typically performed endoscopically and is based on the principle of restoring patency (i.e., the condition of not being blocked or obstructed) of the sinus ducts and ostia by enlarging the opening and allowing the clearance of mucus from the sinus into the nose to resume. The development of endoscopic sinus surgery now allows sinus surgery to be performed from an intranasal approach, thus eliminating the need for external incisions. Endoscopic sinus surgery is commonly done with the use of thin fiber-optic tools, which allow visualization and manipulation of the surgical site without the need for surgical incisions in the mouth or face. Once the endoscopic tools are in place in the surgical site, small tools are typically used to obliterate the sinus tissue and bone to open the sinus passages.

More recently, a technique commonly referred to as balloon catheterization or sinuplasty has been proposed as an alternative to standard endoscopic surgery. Sinuplasty is a minimally invasive surgical procedure that has been used to effectively treat sinusitis while minimizing the amount of trauma experienced by the patient during and after surgery. Because the procedure is less invasive than other surgical techniques, sinuplasty promotes faster healing, less postoperative care, minimal pain and bleeding, and improved quality of life for many patients who suffer with chronic sinusitis.

A variety of proposals have previously been made for the treatment of sinusitis and other disorders of the ear, nose, throat and paranasal sinuses. For example, sinus guiding catheters, sinus guide wires, and sinus balloon catheters and other devices useable to perform minimally invasive, minimally traumatic ear, nose and throat surgery have previously been described in U.S. patent application Ser. No. 11/116,118 entitled “Methods and Devices for Performing Procedures Within the Ear, Nose, Throat and Paranasal Sinuses,” Ser. No. 10/912,578 entitled “Implantable Device and Methods for Delivering Drugs and Other Substances to Treat Sinusitis and Other Disorders,” Ser. No. 10/829,917 entitled “Devices, Systems and Methods for Diagnosing and Treating Sinusitis and Other Disorders of the Ears, Nose and/or Throat,” Ser. No. 10/912,578 entitled “Implantable Device and Methods for Delivering Drugs and Other Substances to Treat Sinusitis and Other Disorders,” Ser. No. 10/944,270 entitled “Apparatus and Methods for Dilating and Modifying Ostia of Paranasal Sinuses and Other Intranasal or Paranasal Structures” and Ser. No. 11/037,548 entitled “Devices, Systems and Methods For Treating Disorders of the Ear, Nose and Throat.”

Sinuplasty involves positioning an expandable dilation device, such as a deflated balloon, inside the clogged sinus pathway and dilating the balloon in order to open the clogged pathway. Fluoroscopy is typically used intermittently during the procedure to confirm completion of the individual steps, being careful to minimize the total dose of radiation delivered. The guide catheter is typically introduced into the nasal cavity, under endoscopic visualization, and placed adjacent to the obstructed sinus opening or ostium. A flexible guide wire is then introduced through the guiding catheter until the tip of the wire rests near the obstructed sinus ostium. Using fluoroscopy the guide wire is advanced through the obstructed sinus ostium. Then a balloon catheter is advanced over the wire, positioned within the ostium and dilated. Thereafter, the catheter was removed and the dilated ostium was inspected endoscopically.

In other embodiments, using fluoroscopic imaging, a small flexible wire is guided into the sinus. Over this guide wire, a dilation balloon is passed into the sinus cavity. Once the balloon catheter is in positioned inside the clogged pathway, the balloon is dilated in order to open the clogged pathway. Typically balloon inflation is accomplished by injecting a fluid into the balloon catheter. The catheter is subsequently removed, and the dilated opening is inspected.

The use of malleable materials in the construction of guide catheters and guide wire devices has been disclosed in the prior art. Such embodiments typically include a region which allows the guide wire or guide catheter to be shaped prior to insertion. For example, U.S. patent application Ser. No. 11/116,118 entitled “Methods and Devices for Performing Procedures Within the Ear, Nose, Throat and Paranasal Sinuses,” discloses embodiments of guide catheters comprised of a tube made from a malleable material. However, the disclosed catheters include either preformed bends or are malleable only at the distal end. The Ser. No. 11/116,118 Application also discloses an embodiment comprised of a malleable guide wire, which may be custom shaped prior to insertion, over which the body of the working catheter device may be guided into the sinus ostium or duct, or a sinus cavity.

U.S. patent application Ser. No. 11/347,147 entitled “Balloon Catheters and Methods for Treating Paranasal Sinuses,” discloses a balloon catheter comprised of a single tube formed of a malleable material, such as stainless steel. The disclosed balloon catheter does not require a guide catheter or guide wire device to access a sinus ostium or sinus cavity, in that the malleable hypotube is of sufficient stiffness and column strength to act as a pushable member to be pushed through a surgically prepared small, tight opening from a sinus into the nose, through a sinus ostium or duct, or into a sinus cavity. However, the catheter disclosed in Ser. No. 11/347,147 application appears to be constructed of a single tube of uniform malleability. Moreover, only a single preformed curve or bend near the distal end of the catheter tube is demonstrated. It has been found that such catheters, while stiff enough to reach the sinus ostia are sometimes not flexible enough to prevent puncturing the ostia. Conversely, while a catheter of uniform stiffness may be flexible enough to place the dilation means into the ostia, it may be too flexible to navigate the tortuous nasal anatomy. Thus, there remains a need in the art for further development and refinement of balloon catheters (and other dilator devices) for use in dilating the ostia of paranasal sinuses.

### SUMMARY OF THE INVENTION

The present invention overcomes many of the disadvantages of prior art sinuplasty devices by providing an improved balloon catheter whose shape may be adjusted more easily prior to insertion and positioning in a clogged sinus pathway, and without using a pre-positioned guide catheter or guide wire device.

The improved balloon catheter includes a first tubular segment, which has multiple zones of differing malleability along its length. The catheter further includes a coaxially aligned hypotube formed of a malleable material, which is positioned within the lumen of the first tubular segment and extends from the distal end of the first tubular segment. In one embodiment the hypotube runs the length of the first tubular segment; while in another embodiment, the hypotube runs along only a portion of the length of the first tubular segment. A portion of the outer circumferential surface of the hypotube is permanently affixed and sealed to an inner circumferential surface of the first tubular segment in the vicinity of the distal end of the first tubular segment. The outer circumference of the distal end of the first tubular segment is gently tapered along its length so as to smoothly transition to the circumference of the hypotube. The hypotube terminates with an atraumatic tip and includes a balloon dilator affixed near the tip. An aperture near the tip of the hypotube fluidly connects the interior of the balloon with the lumen of the hypotube enabling the balloon to selectively expand and contract.

The improved catheter may also include a soft plastic grip around a portion of the first tubular segment. An adapter device, e.g., luer, hub, or manifold, may also be attached to the proximal end of the first tubular segment of the catheter. An inflation device (not shown) may be attached to the adapter device and used to inflate and deflate the balloon on the distal end of the catheter via the lumen of hypotube alone or via the lumen of the hypotube and the lumen of the first tubular segment. The adapter device may also include wings to enable a user to better manipulate the improved catheter.

Further in accordance with the present invention, there is provided a method for dilating an opening of a paranasal sinus. This method generally comprises the steps of; (A) providing an improved catheter of the present invention as described previously; (B) hand shaping the malleable first tubular segment to a desired shape; (C) inserting the improved catheter into the nose, paranasal sinuses or other anatomical structures of the ear, nose or throat; (D) manipulating the improved catheter so as to position the balloon attached to the hypotube into an ostia; and (E) inflating the balloon to dilate the ostia.

### BRIEF DESCRIPTION OF THE DRAWINGS

A more complete understanding of the method and apparatus of the present invention may be had by reference to the following detailed description when taken in conjunction with the accompanying drawings, wherein:

FIG. 1 is a perspective view of the improved catheter of the present invention before it is bent;

FIG. 2a is a cross-sectional schematic view of a first embodiment of the improved catheter of the present invention, wherein the hypotube extends along only a portion of the length of the first tubular segment;

FIG. 2b is a cross-sectional schematic view of a second embodiment of the improved catheter of the present invention, wherein the hypotube extends along the entire length of the first tubular segment;

FIG. 3a is a close-up schematic view of the proximal end of the embodiment of the improved catheter of the present invention shown in FIG. 2a;

FIG. 3b is a close-up schematic view of the proximal end of the embodiment of the improved catheter of the present invention shown in FIG. 2b;

FIG. 3c is a close-up schematic view of the tapered transition segment of the improved catheter of the present invention;

FIG. 3d is a close-up schematic view of the tip of the distal segment of the improved catheter of the present invention; and

FIG. 4 is a flowchart depicting the surgical method that utilizes the improved catheter of the present invention.

Where used in the various figures of the drawing, the same numerals designate the same or similar parts. Furthermore, when the terms “top,” “bottom,” “first,” “second,” “upper,” “lower,” “height,” “width,” “length,” “end,” “side,” “horizontal,” “vertical,” and similar terms are used herein, it should be understood that these terms have reference only to the structure shown in the drawing and are utilized only to facilitate describing the invention.

All figures are drawn for ease of explanation of the basic teachings of the present invention only; the extensions of the figures with respect to number, position, relationship, and dimensions of the parts to form the preferred embodiment will be explained or will be within the skill of the art after the following teachings of the present invention have been read and understood. Further, the exact dimensions and dimensional proportions to conform to specific force, weight, strength, and similar requirements will likewise be within the skill of the art after the following teachings of the present invention have been read and understood.

### DETAILED DESCRIPTION OF THE INVENTION

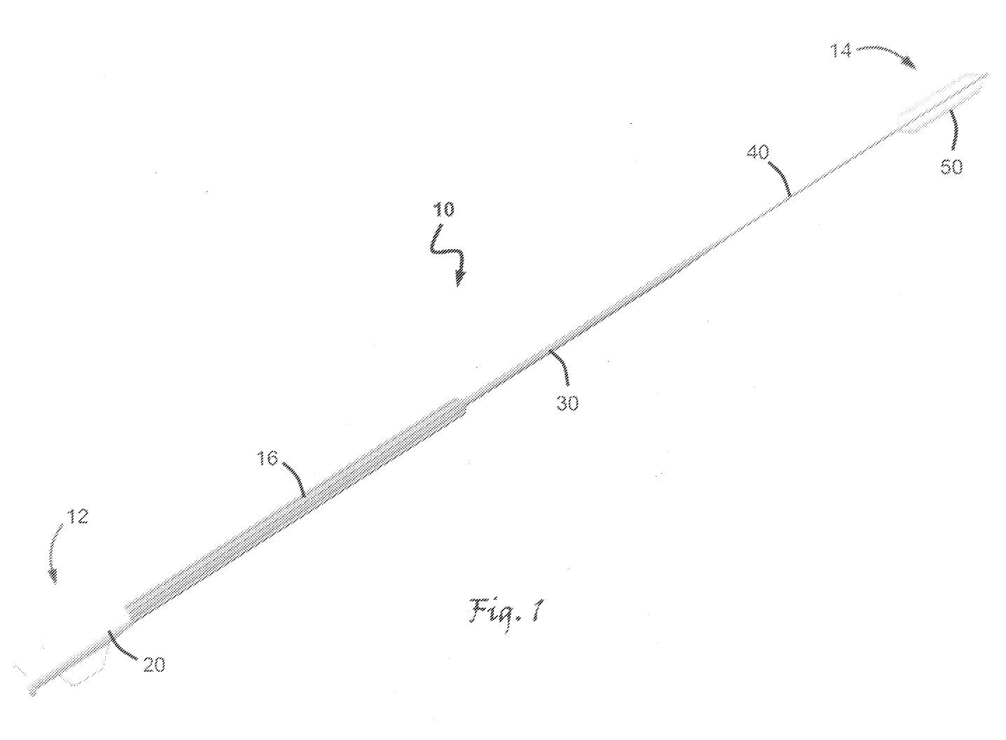

With reference to FIG. 1, a perspective view of an embodiment of the improved catheter 10 of the present invention is shown. The improved catheter 10 is depicted in its non-deformed state prior to surgery. The improved catheter 10 includes a first tubular segment 30, which has multiple zones of differing malleability along its length. The catheter 10 further includes a coaxially aligned second tubular segment or hypotube 40 formed of a malleable material, which is positioned within the lumen 33 of the first tubular segment 30 and extends away from the distal end 32 of the first tubular segment 30. As will be shown in greater detail, a portion of the outer circumferential surface of the hypotube 40 is permanently affixed and sealed to an inner circumferential surface of the first tubular segment 30 in the vicinity of the distal end of the first tubular segment 30. The outer circumference of the distal end of the first tubular segment 30 is gently tapered along its length so as to smoothly transition to the circumference of the hypotube 40. The hypotube 40 terminates with an atraumatic distal tip 48 and includes a balloon dilator 50 affixed near the tip 46. An aperture or port 46 near the tip 48 of the hypotube 40 fluidly connects the interior of balloon 50 with the lumen 43 of the hypotube 40 enabling the dilation means 50 to be selectively dilated.

An adapter device 20 may be attached to the proximal end 12 of the catheter 10. While adapter device 20 is depicted in FIG. 1 as a conventional luer device, it is understood that adapter device 20 may alternatively comprise a conventional hub or manifold device. An inflation device (not shown) may be attached to the adapter device 20 and used to inflate and deflate the balloon dilator 50 on the distal end 14 of the catheter 10 via the lumen 43 of hypotube 40 alone, or in conjunction with the lumen 33 of the first tubular segment 30. The adapter device may also include wings to enable a user to better manipulate the improved catheter. The catheter 10 may also include a soft plastic grip 16 around a portion of the first tubular segment 30.

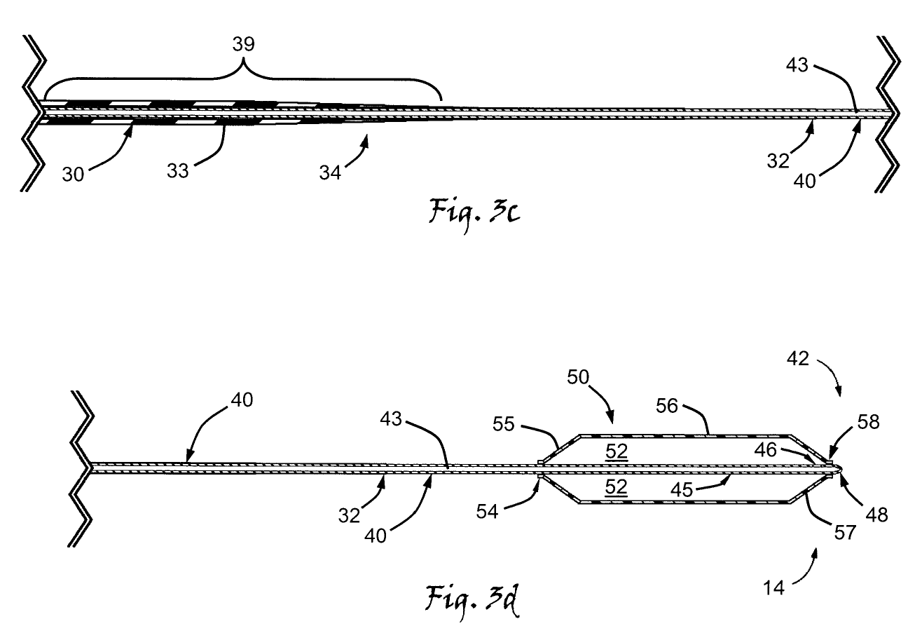

With reference now to the Figures and in particular the close-up schematic views depicted in FIGS. 3a-3d, a more thorough description of the improved catheter device of the present invention will be described. All illustrations of the improved catheter 10 in the Figures are depicted in its non-deformed state prior to surgery. The shape of the first tubular segment 30 may be adjusted as determined by the judgment of the surgeon and the individual anatomy of the patient. The surgeon is able to bend the first tubular segment 30 into the shape of the specific sinus or other passageway through which the improved catheter 10 will be traveling. This reduces the pressure on the nasal passages that typically occurs during endoscopic balloon catheter surgery. It also allows the surgeon to customize the catheter shape to each individual patient's unique anatomy.

The improved catheter 10 includes a first tubular segment 30, which has multiple zones of differing malleability along its length. The first tubular segment 30 is made of any malleable material such as a plastic, metal or a combination thereof, having physical properties that allow the shaft to be bent by hand and retain its shape. For example, in one embodiment, the first tubular segment 30 is comprised of annealed stainless steel tubing having a carbon content sufficient to be hand shapeable by the user. In addition, at least a length (i.e., a zone) of the annealed stainless steel tubing has a carbon content sufficient to provide adequate stiffness to maintain the preset shape when navigating a nasal cavity. In a preferred embodiment, the first tubular segment 30 is comprised of an annealed stainless steel tube approximately 175 mm in length and having a maximum outside diameter of approximately 1.57 mm±0.05 mm, an inside diameter of approximately 0.062 mm±0.002 mm and a wall thickness of approximately 0.010 inches±10%.

A key aspect of the present invention is that the malleability of the first tubular segment 30 is not uniform along its length, but is comprised of multiple zones of differing malleability. By enabling the malleability of the first tubular segment 30 to vary along its length, the design strength and flexibility characteristics of the catheter 10 can be optimized for a particular application.

For example, the first tubular segment 30 may be designed to include a first zone 37 near its proximal end 32 that is relatively stiff and having relatively low malleability to improve its ability to transmit longitudinal forces; a second intermediate zone 38 having a relatively higher malleability which is optimized to transmit rotational displacements while maintaining its cross sectional profile; and a third zone 39 that is more flexible and having a relatively high malleability to improve its ability to travel through intricate anatomical passageways. It will also be observed that the length of the various zones may vary. Moreover, a zone may be designed so that the malleability gradually increases or attenuates within the particular zone. While the foregoing is a relatively simple example, it will be seen that by varying the malleability characteristics of the first tubular segment 30 along its length, a catheter may be optimized for its intended application.

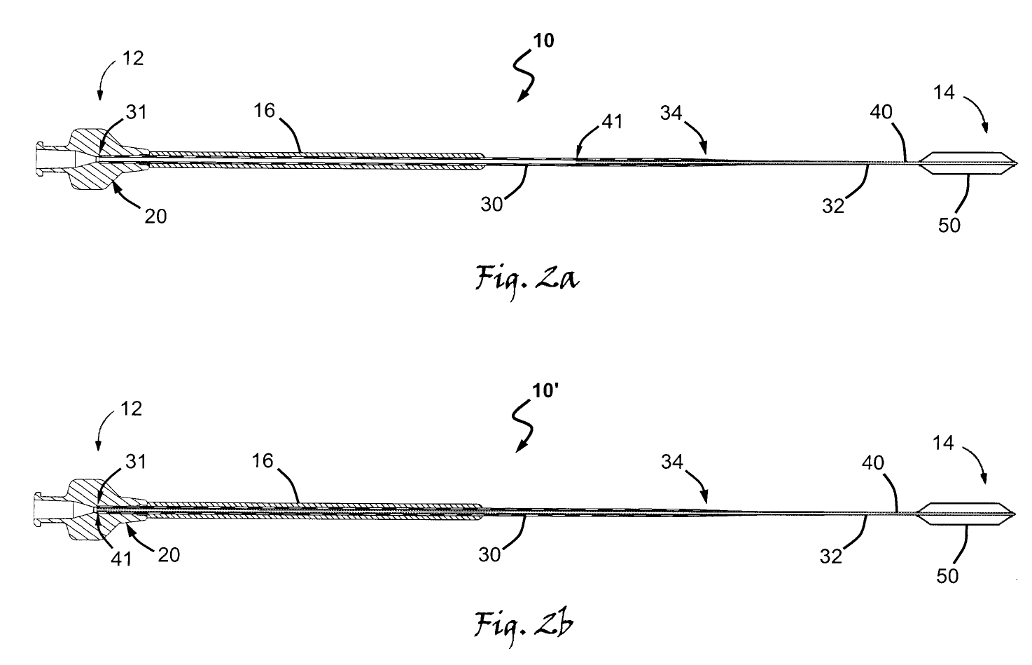

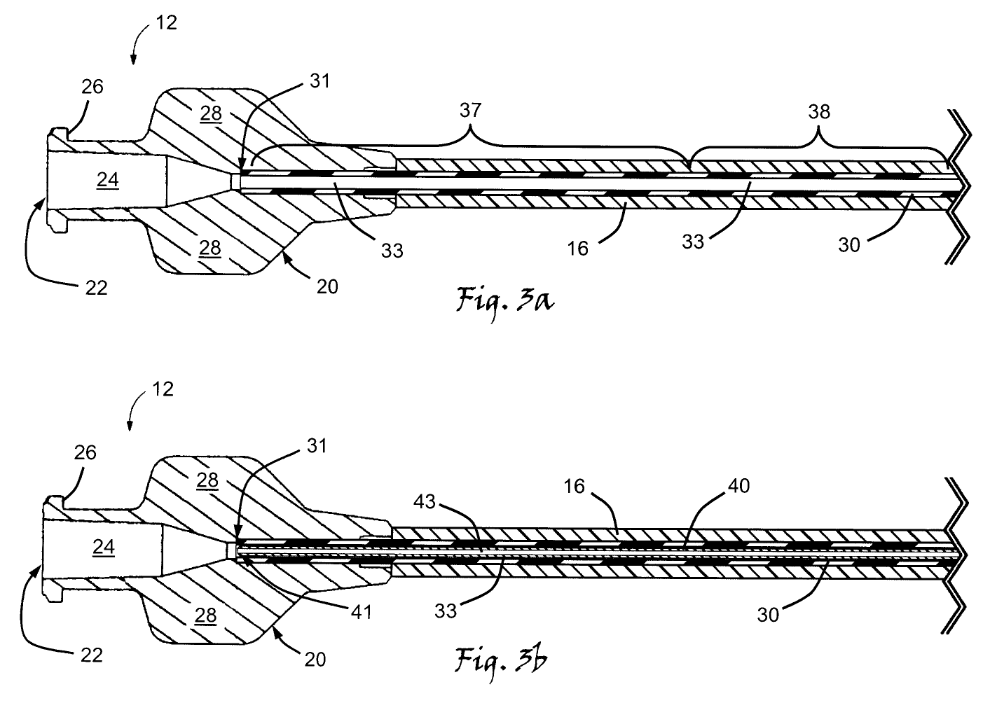

The catheter 10 further includes a coaxially aligned second tubular segment or hypotube 40 formed of a malleable material, which is positioned within the lumen 33 of the first tubular segment 30 and extends away from the distal end 32 of the first tubular segment 30. The overall length of the hypotube 40 may vary depending upon the specific application. For example, as shown in FIGS. 2b and 3b, in one embodiment of the improved catheter 10′ the hypotube 40 runs the length of the first tubular segment. In contrast, as shown in FIGS. 2a and 3a, in another embodiment of the improved catheter 10, the hypotube 40 runs along only a portion of the length of the first tubular segment.

The hypotube 40 is made of any malleable material such as a plastic, metal or a combination thereof, and has a malleability greater than that of any zone in the first tubular segment 30. For example, in one embodiment, the hypotube 40 is comprised of non-annealed stainless steel tubing having an outside diameter of approximately 0.57±0.05 mm, and an inside diameter of approximately 0.0115 mm-0.0130 mm.

As shown in FIG. 3c, a portion of the outer circumferential surface of the hypotube 40 is permanently affixed and sealed to an inner circumferential surface of the first tubular segment 30 in the vicinity of the distal end 32 of the first tubular segment 30. The hypotube 40 is bonded to the first tubular segment 30 via glue, welding, swaging, or friction fit. In one embodiment, the bonding is designed to withstand 15 atm of internal pressure without leaking.

With reference again to FIG. 3c, it will be observed that the first tubular segment 30 includes a tapered region 34 prior to its distal end 32 wherein the outer circumference of the distal end of the first tubular segment 30 is gently tapered along its length so as to smoothly transition to the circumference of the hypotube 40. The tapered region reduces friction within the anatomical environment and enables the improved catheter 10 to more easily travel through complex nasal passageways. In a preferred embodiment, the tapered region 34 may be designed so that the distal end 32 of the first tubular segment 30 extends past the proximal neck 54 and into the interior of the dilation balloon 50.

With reference now to FIG. 3d, the hypotube 40 terminates with an atraumatic distal tip 48 and includes a balloon dilator 50 affixed near the tip 48. A portion of the hypotube 40 may be include a coating to aid bonding with the balloon. For example, the coating may comprise a nylon or UV activated glue. The tip 48 is sealed closed with a full radius seal and is free of oxide stains, burrs or other debris. An aperture or port 46 near the tip 48 of the hypotube 40 fluidly connects the interior 52 of balloon 50 with the lumen 43 of the hypotube 40 enabling the dilation means 50 to be selectively dilated. For example, in one embodiment, the port 46 comprises a circular aperture having a diameter of 0.254 mm and positioned approximately 8 mm from the atraumatic distal tip 48.

The dilation means or balloon 50 is constructed of an elastic material (preferably nylon) and has a length of approximately 4 mm to 30 mm, preferably 22 mm, and a working inflated diameter of 2 mm to 10 mm, preferably 7 mm, for use in the sinus system, except for use in the nasofrontal duct where the preferable inflated working diameter is 5 mm. The balloon has a proximal neck 54, a proximal tapered region 55, a center region 56, a distal tapered region 57, and a distal neck 58. In accordance with conventional procedures, the balloon 50 is situated over a distal segment 45 of hypotube 40 that includes an aperture or port 46 which fluidly connects the interior 52 of the balloon with the lumen 43 of the hypotube 40 enabling the balloon 50 to selectively expand and contract. The distal neck 58 of the balloon 50 is generally aligned with the distal end 42 of hypotube 40. The proximal and distal necks 54, 58 are bonded and sealed to the exterior surface of the hypotube 40. An adhesive, such as cyanoacrylate, may be used to bond and seal the necks of the balloon 50 to the exterior surface of the hypotube 40. Alternatively, the necks 54, 58 may be bonded to the exterior surface of the hypotube 40 by means of laser weld or thermo bond.

With reference again now to the Figures and in particular the close-up schematic views depicted in FIGS. 3a-3d, the improved catheter 10 of the present invention may include adapter device 20 which is attached onto the proximal end 31 of the first tubular segment 30. The adapter device 20 typically includes an inlet 22 and passageway 24 through which the lumens 33, 43 of the first tubular segment 30 and the hypotube 40 may be accessed. The adapter device 20 may also include a flange 26 surrounding the inlet 22. While adapter device 20 is depicted in the Figures as a conventional luer device, it is understood that adapter device 20 may alternatively comprise a conventional hub or manifold device. An inflation device (not shown) may be attached to the adapter device 20 and used to inflate and deflate the balloon dilator 50 on the distal end 14 of the catheter 10 via the lumen 43 of hypotube 40, either alone, or in conjunction with the lumen 33 of the first tubular segment 30. The adapter device 10 may also include wings 28 to enable a user to better manipulate the improved catheter 10. The catheter 10 may also include a soft plastic grip 16 around a portion of the first tubular segment 30 to assist the user in manipulating the device.

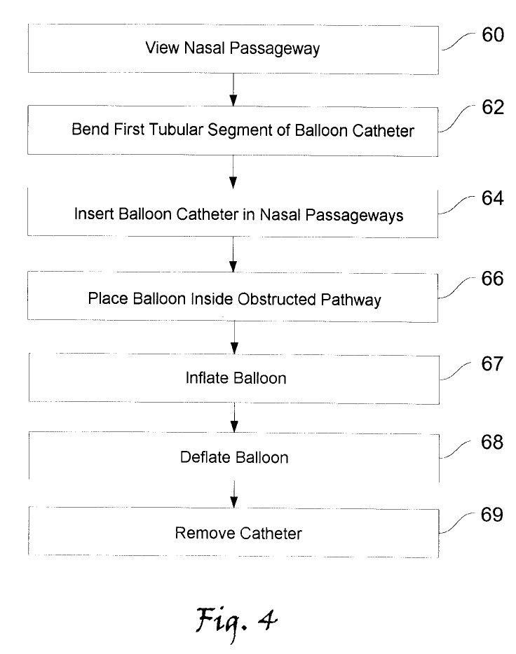

The endoscopic surgical method utilizing the improved balloon catheter of the present invention is depicted in the flow chart in FIG. 4. First, the surgeon uses an imaging device, MRI, CT or other image guidance means to view the nasal passageway 60 and determine the path to the region inside the patient's body causing sinusitis. The surgeon uses this information to bend 62 the malleable first tubular section into a shape consistent with the path that will be followed by the improved balloon catheter. Next, the surgeon inserts the improved balloon catheter into the patient's nasal cavity 64 and guides it into the affected region 66. The surgeon verifies placement of the balloon using an imaging device. Finally, the surgeon inflates the balloon 67 for a predetermined period of time, deflates the balloon 68, and removes the improved catheter of the present invention 69 from the sinus system.

It will now be evident to those skilled in the art that there has been described herein an improved balloon catheter whose shape may be adjusted more easily and efficiently prior to insertion and positioning in a clogged sinus pathway, and without using a pre-positioned guide catheter or guide wire device.

Although the invention hereof has been described by way of a preferred embodiment, it will be evident that other adaptations and modifications can be employed without departing from the spirit and scope thereof. For example, the actual dimensions and materials employed could be varied. In addition, the first and second tubular segments may each have more than one lumen. Moreover, a wide variety of dilation means could be adapted to the improved catheter device of the present invention. The terms and expressions employed herein have been used as terms of description and not of limitation; and thus, there is no intent of excluding equivalents, but on the contrary it is intended to cover any and all equivalents that may be employed without departing from the spirit and scope of the invention.

We claim:

1. A balloon catheter for treating paranasal sinus airways, comprising:
a first tubular segment comprising a first proximal end, a distal end, and a first lumen extending therethrough, wherein said first tubular segment has multiple zones of differing malleability along its length;
a second tubular segment comprising a second lumen extending through at least a portion of the length of said second tubular segment, wherein said second tubular segment is attached to and extends away from said distal end of said first tubular segment, wherein said second tubular segment is co-axially aligned with said first lumen and includes a distal segment which terminates with an atraumatic distal tip, said distal segment include an aperture which opens said second lumen to the exterior of said second tubular segment; and
a dilation means attached to said distal segment of said second tubular segment, wherein said dilation means is selectively inflated and deflated by means of said aperture.

2. The balloon catheter of claim 1, wherein a portion of said second tubular segment runs the length of said first lumen.

3. The balloon catheter of claim 1, wherein said second tubular segment runs only a portion of the length of said first lumen.

4. The balloon catheter of claim 1, wherein the distal end of said first tubular segment is tapered along its length.

5. The balloon catheter of claim 1, wherein said second tubular segment is attached to said first tubular segment by adhesive means.

6. The balloon catheter of claim 1, wherein said second tubular segment is attached to said first tubular segment by laser welding.

7. The balloon catheter of claim 1, wherein said first tubular segment comprises annealed metal tubing.

8. The balloon catheter of claim 7, wherein said first tubular segment comprises annealed stainless steel tubing.

9. The balloon catheter of claim 8, wherein said first tubular segment has an outside diameter of approximately 1.57 mm±0.05 mm, an inside diameter of approximately 0.062 mm±0.002 mm and a wall thickness of approximately 0.010 inches±10%.

10. The balloon catheter of claim 8, wherein said second tubular segment comprises non-annealed stainless steel tubing having a malleability greater than said first tubular segment.

11. The balloon catheter of claim 10, wherein said second tubular segment has an outside diameter of approximately 0.57±0.05 mm, and an inside diameter of approximately 0.0115 mm-0.0130 mm

12. The balloon catheter of claim 1, further comprising an adapter device attached to said first proximal end of said first tubular segment, wherein said adapter device includes an inlet and passageway to said first lumen.

13. The balloon catheter of claim 1, further comprising a soft plastic grip around a portion of said first tubular segment.

13. The balloon catheter of claim 1, wherein said dilation means comprises a balloon constructed of a nylon.

14. The balloon catheter of claim 13, wherein said balloon inflates to a maximum diameter of approximately 5 mm.

15. The balloon catheter of claim 13, wherein said balloon inflates to a maximum diameter of approximately 7 mm.

16. The balloon catheter of claim 13, wherein said balloon inflates to a maximum diameter of approximately 9 mm.

17. A balloon catheter for treating paranasal sinus airways, comprising:
a first tubular segment comprising a first proximal end, a distal end, and a first lumen extending therethrough, wherein said first tubular segment includes a plurality of zones of differing malleability along its length;
a second tubular segment comprising a second lumen extending through at least a portion of the length of said second tubular segment, wherein said second tubular segment is attached to and extends away from said distal end of said first tubular segment, wherein said second tubular segment is co-axially aligned with said first lumen and includes a distal segment which terminates with an atraumatic distal tip, said distal segment include an aperture which opens said second lumen to the exterior of said second tubular segment; and
a dilation means attached to said distal segment of said second tubular segment, wherein said dilation means is selectively inflated and deflated by means of said aperture.

18. The balloon catheter of claim 17, wherein said plurality of zones comprises 3 zones.

19. The balloon catheter of claim 17, wherein said plurality of zones comprises 4 zones.

20. The balloon catheter of claim 17, wherein said plurality of zones comprises 5 zones.

21. An endoscopic surgical method, comprising the steps of:
(a) providing a human body with a nasal system having at least one obstructed fluid pathway;
(b) providing a balloon catheter having a first and second tubular segments, wherein said tubular segments are attached and wherein said first tubular segment has multiple zones of differing malleability along its length;
(c) viewing the nasal system with an imaging device;
(d) bending the first tubular segment into a shape approximating the nasal system;
(e) inserting the balloon catheter into the nasal system;
(f) guiding the balloon catheter to the obstructed fluid pathway;
(g) inserting the balloon catheter into the obstructed fluid pathway;
(h) inflating the balloon catheter;
(i) deflating the balloon catheter; and
(j) removing the balloon catheter from the nasal system.
